(19) United States
(12) Patent Application Publication
(10) Pub. No.: US 2010/0029978 A1
(43) Pub. Date: Feb. 4, 2010

(54) RAPIDLY CURING CYANOACRYLATES AS ADHESIVES

(76) Inventors: Noeleen Swords, Kiltipper (IE); Louise Gilbride, Dublin (IE); Colette Hoare, Dublin (IE)

Correspondence Address: HENKEL CORPORATION, One Henkel Way, ROCKY HILL, CT, 06067, US

(21) Appl. No.: 12/578,923

(22) Filed: Oct. 14, 2009

Related U.S. Application Data
(63) Continuation of application No. PCT/EP2008/054256, filed on Apr. 9, 2008.

(30) Foreign Application Priority Data
Apr. 18, 2007 (EP) 07007847.2

(51) Int. Cl. C07C 255/03

(52) U.S. Cl. 558/381

(57) ABSTRACT

The present invention relates to a polymerizable adhesive composition which comprises, at least as one constituent, a cyanacrylate component and which requires a comparitively short time for curing when used on surfaces. The present invention therefore also includes a method for the production of the cyanacrylate component described above, and the cyanacrylate component as such.

### CROSS REFERENCE TO RELATED APPLICATIONS

This application is a continuation under 35 U.S.C. Sections 365(c) and 120 of International Application No. PCT/EP2008/054256, filed Apr. 9, 2008 and published on Dec. 4, 2008 as WO 2008/128888, which claims priority from European Patent Application No. 07007847.2 filed Apr. 18, 2007, which are incorporated herein by reference in their entirety.

The present invention relates to a polymerizable adhesive composition that encompasses a cyanoacrylate component as at least one constituent and exhibits no overstabilization by way of a polymerization inhibitor, so that a comparatively short time is required for curing upon application onto surfaces. The present invention therefore also contains a method for manufacturing the above-described cyanoacrylate component, as well as the cyanoacrylate component as such.

### BACKGROUND OF THE INVENTION

Because of their ease of application and rapid curing rate, and the strength of the resulting adhesive bond, cyanoacrylate-based polymerizable monomeric adhesive compositions have become widely used in both industrial and medical applications. It is known that monomeric forms of cyanoacrylates are extremely reactive and polymerize rapidly in the presence of even very small quantities of a polymerization initiator, including moisture contained in the air or present on surfaces. Polymerization is initiated by anions, free radicals, zwitterions, or ion pairs. Once polymerization has been started, the curing rate can be very high. Cyanoacrylate-based polymerizable monomeric adhesive compositions have therefore proven to be attractive solutions, for example, for joining plastics, rubber, glass, metals, wood, and more recently also biological tissues. Medical applications of cyanoacrylate-based monomeric adhesive compositions include both utilization as alternatives to or in addition to surgical sutures and staples when closing wounds, and utilization to cover and protect superficial wounds such as lacerations, abrasions, burns, stomatitis, inflammations, and other open superficial wounds.

The U.S. Pat. No. 5,328,687 by Leung et al., U.S. Pat. No. 3,527,841 by Wicker et al., U.S. Pat. No. 3,722,599 by Robertson et al., U.S. Pat. No. 3,995,641 by Kronenthal et al., and U.S. Pat. No. 3,940,362 by Overhults, for example, disclose monomeric cyanoacrylates that are suitable as surgical adhesive agents.

In the context of medical utilization of a cyanoacrylate-based adhesive composition, application is usually accomplished in monomeric form. Subsequent anionic in-situ polymerization directly on the tissue surface then causes wound adhesion or coverage.

As compared with the utilization of sutures or staples for wound care, the alternative use of cyanoacrylate-based wound adhesives offers a number of advantages. Wound sutures in the direct vicinity of the injury being treated cause additional injuries because of the penetration of the needle into the tissue and the need in some cases to administer an anesthetic, and require a time-consuming procedure for application. The same is true of wound treatment using staples. The result is that the use of these agents presents problems especially in pediatric cases, since because of the adverse effects associated with them, they trigger severe anxiety and aversion reactions in the often very young patients.

The problems set forth above can be at least partially circumvented or mitigated by the inherently painless application of a cyanoacrylate-based wound adhesive in accordance with a method described by Halpern in U.S. Pat. No. 3,667,472 or by Banitt et al. in U.S. Pat. No. 3,559,652.

Despite these advantages, the medical use of cyanoacrylate-based adhesive compositions can be associated with certain problems, since it is known that both the monomers and the polymer that is formed can bring about serious irritation of the tissue in the application area. This negative tissue reaction is attributed principally to the biological breakdown process of the polymer that takes place in vivo, which, as described in the following citations—F. Leonard et al., Journal of Applied Polymer Science, Vol. 10, pp. 259-272 (1966); F. Leonard, Annals New York Academy of Sciences, Vol. 146, pp. 203-213 (1968); Tseng, Yin-Chao, et al., Journal of Applied Biomaterials, Vol. 1, pp. 111-119 (1990), and Tseng, Yin-Chao, et al., Journal of Biomedical Materials Research, Vol. 24, pp. 1355-1367 (1990)—leads to the release of formaldehyde.

A number of structural modifications have therefore been made in the past in order to enhance the biocompatibility of cyanoacrylate-based adhesives. By extending the alkyl chain in the cyanoacrylate ester, for example, it has been possible to greatly reduce the speed of the biological breakdown process and thus the rate of formaldehyde release into the affected tissue. Whereas short-chain cyanoacrylate esters (e.g. methyl-2-cyanoacrylate) are subject to rapid biodegradation, the longer-chain analogs such as, for example, butyl-2-cyanoacrylate, octyl-2-cyanoacrylate, or decyl-2-cyanoacrylate are notable for a much reduced breakdown rate.

As described in U.S. Pat. No. 6,667,031 by M. Azevedo, the synthesis of cyanoacrylate monomers is based on thermal cracking, at temperatures from 150 to more than 200° C., of the prepolymer produced upon the reaction of cyanoacetate with formaldehyde, and subsequent separation of the resulting monomers from the reaction solution by distillation. Thermal depolymerization is successful only when this process occurs in the presence of stabilizers, or mixtures of stabilizers, that can prevent both radical and anionic repolymerization of the resulting monomers under the reaction conditions described. As disclosed in U.S. Pat. Nos. 3,559,652 and 5,582,834, the radical stabilizers are, by way of example, hydroquinone, hydroquinone monomethyl ether, nitrohydroquinone, catechol and hydroquinone monomethyl esther. Anionic polymerization inhibitors are as a rule, but not exclusively, Lewis acids such as, for example, sulfur dioxide, nitrogen monoxide, or boron trifluoride, or inorganic or organic Brønstedt acids such as, for example, sulfuric acid, phosphoric acid, or sulfonic acids.

Determining the optimum concentration of the anionic polymerization inhibitor represents a difficult technical problem. Under the drastic conditions of thermal depolymerization of the prepolymer, too low a concentration results in significant repolymerization of the monomers that have already formed. A very high concentration of the anionic stabilizer, on the other hand, causes a portion of the stabilizer to be carried over from the reaction solution upon distillative separation of the monomer. This results in a residual concentration of the anionic stabilizer in the distilled cyanoacrylate monomer, which is responsible for an overstabilization of the product so that effective polymerization of the cyanoacrylate monomer on the tissue surface is later inhibited.

The problem of a high residual concentration of an anionic stabilizer is especially important particularly in the production of long-chain high-boiling monomeric cyanoacrylate esters such as, for example, octyl-2-cyanoacrylate or decyl-2-cyanoacrylate. As compared with short-chain cyanoacrylate esters, separating out from the reaction solution the particular monomer that has been produced requires higher distillation temperatures and lower distillation pressures. As an undesired side effect of this, a portion of the anionic stabilizer is carried over into the monomeric product, resulting in an overstabilization of the long-chain biocompatible cyanoacrylate ester that is extremely negative for later utilization.

This overstabilization in terms of anionic polymerization affinity can be compensated for by adding polymerization initiators or promoters to the monomeric adhesive composition. It is possible to use as polymerization initiators or promoters, for example, amines that exhibit sufficiently good solubility under the prevailing conditions.

An important consideration with all additives is that, specifically in the medical application sector, the additives must have no toxicologically objectionable effect on the particular organism or on the tissue that has in any event already suffered serious prior damage. Care must therefore be taken in all cases, when developing medical wound adhesives, to limit as much as possible the number of additives contained, in order to minimize risks to the patient.

In this context, U.S. Pat. No. 6,849,082 by M. Azevedo discloses a method for removing the anionic stabilizer from a monomeric adhesive composition prior to application onto the tissue surface. The monomeric adhesive composition is brought directly into contact with a substance for removing the stabilizer (Lewis acid or organic/inorganic Brønstedt acid). Examples of this substance are ion exchangers, molecular sieves, zeolites, chelating agents, activated carbon systems, or other substances of an anionic nature.

A related invention is described by M. Azevedo in U.S. Pat. No. 6,667,031. Here the anionic stabilizer is removed, prior to application of the monomeric adhesive composition, by contact with a silicate, a polyvinylpyrrolidone-based polymer or copolymer, or a polymer that possesses functional groups such as carbonyl, hydroxyl, amide, carboxylate, amine, ether, anhydride, ester, urethane, or sulfone, by the creation of physical interactions such as adsorption or absorption, hydrogen bridge bonds, or the occurrence of a chemical reaction.

The approach common to the methods described above is that overstabilization of the monomeric cyanoacrylate-based adhesive composition is to be counteracted by the addition of an initiator or by way of a purification step, so as thereby to enable effective polymerization on the tissue surface or to increase the polymerization rate. What would be desirable in this context would be a cyanoacrylate-based adhesive composition that, because of its manufacturing process, exhibits such a low concentration of undesirable polymerization inhibitors that overstabilization of the polymerizable adhesive composition does not occur, thereby making possible direct application with no preceding purification steps and without the addition of additives.

The object that accordingly results for the present invention is that of making available a cyanoacrylate-based polymerizable adhesive composition that exhibits no overstabilization resulting from a polymerization inhibitor, so that upon application to surfaces, curing of the adhesive composition occurs within a comparatively short period of time.

It has now been found, surprisingly, that in the context of cyanoacrylate components having an at least 90 wt %, by preference at least 95 wt %, particularly preferably at least 98 wt %, and very particularly preferably at least 99 wt % weight proportion of cyanoacrylate, or of mixtures of a cyanoacrylate with further cyanoacrylates, curing on an ABS surface occurs in less than 80 s without the addition of a polymerization initiator or polymerization accelerator.

Suitable polymerization initiators or polymerization accelerators are well known to one skilled in the art. The addition of these substances or substance mixtures to monomeric cyanoacrylates causes the polymerization process to proceed in accelerated fashion as compared with identical monomeric cyanoacrylates to which the relevant substances or substance mixtures have not been added.

In a preferred embodiment of the present invention, the inventive cyanoacrylate component consists essentially of only the aforementioned cyanoacrylate or a mixture of said cyanoacrylates.

In another preferred embodiment of the present invention, the inventive cyanoacrylate component consists of the inventive cyanoacrylate as well as primary and secondary anionic polymerization inhibitors and optionally at least one free radical chain polymerization inhibitor.

The general structure of the cyanoacrylate according to the present invention is described by formula (I), R being a substituted or unsubstituted, straight-chain, branched or cyclic alkyl group having 5 to 18 C atoms and/or an aromatic group or acyl group.

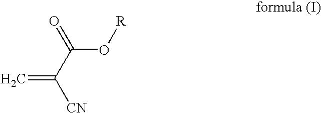

Preferred embodiments encompass, without being limited thereto, n-pentyl 2-cyanoacrylate, iso-pentyl 2-cyanoacrylate (such as 1-pentyl, 2-pentyl, and 3-pentyl), cyclopentyl 2-cyanoacrylate, n-hexyl-2-cyanoacrylate, iso-hexyl 2-cyanoacrylate (such as 1-hexyl, 2-hexyl, 3-hexyl, and 4-hexyl), cyclohexyl 2-cyanoacrylate, n-heptyl 2-cyanoacrylate, isoheptyl 2-cyanoacrylate (such as 1-heptyl, 2-heptyl, 3-heptyl, and 4-heptyl), cycloheptyl 2-cyanoacrylate, n-octyl 2-cyanoacrylate, 1-octyl 2-cyanoacrylate, 2-octyl 2-cyanoacrylate, 3-octyl 2-cyanoacrylate, 4-octyl 2-cyanoacrylate, decyl 2-cyanoacrylate, dodecyl 2-cyanoacrylate. Particularly preferred cyanoacrylates of general formula (I) are n-octyl-2-cyanoacrylate or 2-octyl-cyanoacrylate. Mixtures of said cyanoacrylates are also preferred.

In preferred embodiments of the present invention, the inventive cyanoacrylates may also be combined with other cyanoacrylates. For example, a mixture of at least one of said cyanoacrylates with n-butyl 2-cyanoacrylate, such as a mixture of 2-octyl 2-cyanoacrylate with n-butyl 2-cyanoacrylate is preferred.

In a preferred embodiment of the present invention, the inventive cyanoacrylates of general formula (I) may also be present in essentially monomeric form, i.e., the proportion of the corresponding polymer and/or oligomer is less than 5 wt %, preferably less than 1 wt %, and most preferably less than 0.1 wt %, each based on the total amount of inventive cyanoacrylates of general formula (I).

The cyanoacrylates according to the present invention of formula (I) may be manufactured in accordance with methods that are known in the technical sector. U.S. Pat. Nos. 2,721,858 and 3,254,1 11 disclose methods for manufacturing cyanoacrylates. The cyanoacrylates can be manufactured, for example, by reacting an alkyl cyanoacetate with formaldehyde in a nonaqueous organic solvent and in the presence of a basic catalyst, followed by thermal depolymerization of the anhydrous prepolymer in the presence of a stabilizer. Cyanoacrylate monomers that have been manufactured with a low moisture content and in a manner substantially free of contaminants are preferred for biomedical applications.

The moment at which curing of the adhesive bond has been achieved, is determined with the help of specimen bodies with the dimensions 100 mm×25 mm×2 mm, which have an overlapping bond area of 322.6 mm². The surface used for determining the moment of curing of the adhesive bond is an ABS polymer from Williaam Cox Ireland Ltd. The specimen bodies are joined together after applying the cyanoacrylate component (approximately 10 microliters) to the overlapping bond area. The moment at which curing of the adhesive bond has been achieved, is determined by applying a tensile force that is exerted by a 1-kg weight. When the adhesive bond is capable of withstanding this tensile force for at least 5 s, that moment is defined as the moment of curing.

The stated moment of curing is the arithmetic mean of five determination tests.

In a preferred embodiment of the invention, curing of a sterile cyanoacrylate component according to the present invention on an ABS surface takes place in at most 75 s, by preference at most 50 s, and particularly preferably at most 35 s.

In a further preferred embodiment of the invention, curing of a non-sterile cyanoacrylate component on an ABS surface occurs in at most 50 s, by preference at most 25 s, and particularly preferably at most 15 s.

The moment of curing is determined in each case according to the method described above, by applying a tensile force of 1 kg to the adhesive bond.

The adhesive shear strength on nylon after curing of a sterile cyanoacrylate component according to the present invention is by preference at least 1.6 N/mm², particularly preferably at least 1.8 N/mm², and very particularly preferably at least 2.0 N/mm²₁ and after curing of a non-sterile cyanoacrylate component according to the present invention is by preference at least 1.6 N/mm², particularly preferably at least 1.9 N/mm², and very particularly preferably at least 2.5 N/mm².

The adhesive shear strength is determined with the help of specimen bodies with the dimensions 100 mm×25 mm×2 mm, with a bond overlap area of 322.6 mm². Nylon 101 (type 66, natural) from Industrial Safety Supply Co., CT, USA is used as the surface for determination of the adhesive shear strength. The specimen bodies are joined together after applying the cyano-acrylate component (approximately 10 microliters) to the bond overlap area by using staples (stapling force approximately 45 to 90 N) and curing the cyanoacrylate component at room temperature for up to 24 hours. The adhesive shear strength of the cyanoacrylate component is then determined by applying a tensile force parallel to the bond surface and to the main axis of the specimen by using a tensile tester operated at a test speed of 2 mm/min.

The stated adhesive shear strength is given as the arithmetic mean of five determination tests and is given in N/mm².

A further subject of the present invention is a polymerizable adhesive composition containing an inventive cyanoacrylate component as at least one component.

In a preferred embodiment of the invention, the polymerizable adhesive composition contains at least one inorganic acid as a primary anionic polymerization inhibitor and at least one organic sulfonic acid as a secondary polymerization inhibitor, said sulfonic acid being described by the general formula (II)

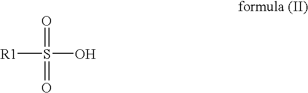

and R1 denoting an unsubstituted aryl group or a mono-, di-, tri-, tetra-, or pentasubstituted aryl group.

In a very particularly preferred embodiment of the invention, R1 in formula (II) is described by the general formula (III), R2 containing a hydrogen atom, a halogen atom, a substituted heteroatom, a substituted or unsubstituted, straight-chain, branched, or cyclic alkyl chain that encompasses 1 to 10 C atoms, or an aromatic group and/or acyl group.

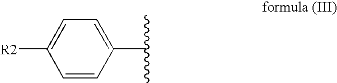

A “heteroatom” is to be understood as any atom that is not carbon or hydrogen.

Particularly preferably R2 stands for a methyl, methoxy, ethyl, ethoxy, n-propyl, isopropyl, or n-butyl group, in particular for a methyl group.

In a preferred form of the invention, the primary anionic polymerization inhibitor is an oxoacid, halogen acid or Lewis acid or a combination of said acids. Particularly preferred exemplary embodiments contain, but are not limited to, sulfur dioxide, boron trifluoride, nitrous oxide, hydrogen fluoride, hydrochloric acid, sulfuric acid, phosphoric acid, perchloric acid or phosphorus pentoxide, or combinations of said acids.

The aforementioned polymerization inhibitors inhibit the polymerization. The primary anionic polymerization inhibitors may optionally also exert a catalytic function in thermal depolymerization of the prepolymer in addition to having a stabilizing effect and/or may neutralize the bases used in synthesis of the prepolymer.

The quantity of primary anionic polymerization inhibitor for the liquid phase and for the vapor phase for stabilization of the polymerizable adhesive composition depends on the type of the particular inhibitors used and on the monomer to be stabilized and can be ascertained by an average person skilled in the art using known techniques.

In a preferred embodiment of the polymerizable adhesive composition according to the present invention, the proportion of the secondary anionic polymerization inhibitor, based on the cyanoacrylate according to the present invention in accordance with formula (I) or on the mixture of a cyanoacrylate according to the present invention in accordance with formula (I) with further cyanoacrylates according to the present invention in accordance with formula (I), is less than 150 ppm, preferably less than 140 ppm, 130 ppm, 120 ppm, 110 ppm, 100 ppm, particularly preferably less than 90 ppm, 80 ppm, 70 ppm, 60 ppm, 50 ppm, very particularly preferably less than 40 ppm, 30 ppm, 20 ppm, and greatly preferably less than 10 ppm.

In a particular embodiment of the invention, the cyanoacrylate component and/or the polymerizable adhesive composition may also have added to it a radical chain polymerization inhibitor, in a concentration easily determined by one skilled in the art. Suitable radical chain polymerization inhibitors are for example phenol compounds, such as hydroquinone, butylated hydroxyanisole (BHA), 2,6-di-tert-butyl-4-methylphenol (BHT), t butyl-catechinone, pyrocatechol, and p-methoxyphenol are usually used. Mixtures of the aforementioned radical chain polymerization inhibitors may also be used. Butylated hydroxyanisole (BHA) is an especially preferred radical chain polymerization inhibitor.

The polymerizable adhesive composition according to the present invention by preference additionally encompasses at least one further component selected from the groups of the plasticizers, thickening agents, antimicrobial active substances, thixotroping agents, skin-care active substances, perfumes, and agents for reducing formaldehyde concentration.

If a plasticizer is present, it imparts flexibility to the polymer formed from the monomer, and by preference contains little or no moisture and should not significantly influence the stability or the polymerization of the monomer. Such plasticizers are useful in polymerized compositions that are to be used to close or cover wounds, incisions, abrasions, inflammations or other applications in which flexibility of the adhesive is desirable.

Triaryl phosphates or trialkyl phosphates and ester compounds are particularly suitable as plasticizers. The alcohol component of the ester involves, by preference, alcohols having 1 to 5, in particular 2 to 4, OH groups and having 2 to 5, in particular 3 or 4 C atoms joined directly to one another. The number of C atoms not directly joined to one another can be up to 110, in particular up to 18 C atoms.

The following substances are suitable as univalent alcohols: methanol, ethanol, 1-propanol, 2-propanol, 1-butanol, 2-butanol, 2,2-dimethyl-1-propanol, 2-methyl-1-propanol, 2,2-dimethyl-1-propanol, 2-methyl-2-propanol, 2-methyl-1-butanol, 3-methyl-1-butanol, 2-methyl-2-butanol, 3-methyl-2-butanol, 1-pentanol, 2-pentanol, 3-pentanol, cyclopentanol, cyclopentenol, glycidol, tetrahydrofurfuryl alcohol, tetrahydro-2H-pyran-4-ol, 2-methyl-3-buten-2-ol, 3-methyl-2-buten-2-ol, 3-methyl-3-buten-2-ol, 1-cyclopropylethanol, 1-penten-3-ol, 3-penten-2-ol, 4-penten-1-ol, 4-penten-2-ol, 3-pentin-1-ol, 4-pentin-1-ol, propargyl alcohol, allyl alcohol, hydroxyacetone, 2-methyl-3-butin-2-ol.

Suitable as divalent alcohols are, for example: 1,2-ethanediol, 1,2-propanediol, 1,3-propanediol, dihydroxyacetone, thioglycerol, 2-methyl-1,3-propanediol, 2-butine-1,4-diol, 3-butene-1,2-diol, 2,3-butanediol, 1,4-butanediol, 1,3-butanediol, 1,2-butanediol, 2-butene-1,4-diol, 1,2-cyclopentanediol, 3-methyl-1,3-butanediol, 2,2-dimethyl-1,3-propanediol, 4-cyclopentene-1,3-diol, 1,2-cyclopentanediol, 2,2-dimethyl-1,3-propanediol, 1,2-pentanediol, 2,4-pentanediol, 1,5-pentanediol, 4-cyclopentene-1,3-diol, 2-methylene-1,3-propanediol, 2,3-dihydroxy-1,4-dioxane, 2,5-dihydroxy-1,4-dithiane.

The following trivalent alcohols may be used: glycerol, erythrulose, 1,2,4-butanetriol, erythrose, threose, trimethylolethane, trimethylolpropane, and 2-hydroxymethyl-1,3-propanediol.

Of the tetravalent alcohols, for example, erythritol, threitol, pentaerythritol, arabinose, ribose, xylose, ribulose, xylulose, lyxose, ascorbic acid, gluconic acid-g-lactone may be used.

Examples of pentavalent alcohols that may be cited are arabitol, adonitol, xylitol.

Further suitable mono- and polyvalent alcohols are familiar to one skilled in the art.

The polyvalent alcohols described above may also be used, for example, in the form of ethers. The ethers can be produced from the aforementioned alcohols, for example, by way of condensation reactions, Williamson ether synthesis, or by reaction with alkylene oxides such as ethylene, propylene, or butylene oxide. Examples that may be cited are: diethylene glycol, triethylene glycol, polyethylene glycol, diglycerol, triglycerol, tetraglycerol, pentaglycerol, polyglycerol, technical mixtures of the condensation products of glycerol, glycerol propoxylate, diglycerol propoxylate, pentaerythritol ethoxylate, dipentaeryrthritol, ethylene glycol monobutyl ether, propylene glycol monohexyl ether, butyldiglycol, dipropylene glycol monomethyl ether.

Monovalent carboxylic acids that may be used for esterification with the aforementioned alcohols are, for example: formic acid, acrylic acid, acetic acid, propionic acid, butyric acid, isobutyric acid, valeric acid, isovaleric acid, 2-oxovaleric acid, 3-oxovaleric acid, pivalic acid, acetoacetic acid, levulinic acid, 3-methyl-2-oxobutyric acid, propiolic acid, tetrahydrofuran-2-carboxylic acid, methoxyacetic acid, dimethoxyacetic acid, 2-(2-methoxyethoxy)acetic acid, pyruvic acid, 2-methoxyethanol, vinylacetic acid, allylacetic acid, 2-pentenoic acid, 3-pentenoic acid.

The following may be mentioned as examples of polyvalent carboxylic acids: oxalic acid, malonic acid, fumaric acid, maleic acid, succinic acid, glutaric acid, acetylenedicarboxylic acid, oxaloacetic acid, acetonedicarboxylic acid, mesoxalic acid, citraconic acid, dimethylmalonic acid, methylmalonic acid, ethylmalonic acid.

Hydroxycarboxylic acids may also be used as starting materials, for example, tartronic acid, lactic acid, malic acid, tartaric acid, citramalic acid, 2-hydroxyvaleric acid, 3-hydroxyvaleric acid, 3-hydroxybutyric acid, 3-hydroxyglutaric acid, dihydroxyfumaric acid, 2,2-dimethyl-3-hydroxypropionic acid, dimethylolpropionic acid, glycolic acid, citric acid.

The esterification can be performed either completely or partially. Mixtures of these acids can also, if applicable, be used for esterification.

The esters produced from these alcohols and carboxylic acids or from the corresponding derivatives are by preference free of catalysts, in particular of alkali metals and amines. This can be achieved by treating the esters according to the present invention with acids, ion exchangers, acetic-acid aluminas, aluminum oxides, activated carbon, or other adjuvants known to one skilled in the art. Distillation can be performed for drying and further purification.

The following may be mentioned as examples of esters particularly suitable as plasticizers: ethyl acetate, butyl acetate, glycerol triacetate, glycerol tripropionate, triglycerol pentaacetate, polyglycerol acetate, diethylene glycol diacetate, 3-hydroxyvaleric acid ethyl ester, lactic acid butyl ester, lactic acid isobutyl ester, 3-hydroxybutyric acid ethyl ester, oxalic acid diethyl ester, mesoxalic acid diethyl ester, malic acid dimethyl ester, malic acid diisopropyl ester, tartaric acid diethyl ester, tartaric acid dipropyl ester, tartaric acid diisopropyl ester, glutaric acid dimethyl ester, succinic acid dimethyl ester, succinic acid diethyl ester, maleic acid diethyl ester, fumaric acid diethyl ester, malonic acid diethyl ester, acrylic acid 2-hydroxyethyl ester, 3-oxovaleric acid methyl ester, glycerol diacetate, glycerol tributyrate, glycerol tripropionate, glycerol dipropionate, glycerol triisobutyrate, glycerol diisobutyrate, glycidyl butyrate, acetoacetic acid butyl ester, levulinic acid ethyl ester, 3-hydroxyglutaric acid dimethyl ester, glycerol acetate dipropionate, glycerol diacetate butyrate, propionic acid butyl ester, propylene glycol diacetate, propylene glycol dibutyrate, diethylene glycol dibutyrate, trimethylolethane triacetate, trimethylolpropane triacetate, trimethylolethane tributyrate, neopentyl alcohol dibutyrate, methoxyacetic acid pentyl ester, dimethoxyacetic acid butyl ester, glycolic acid butyl ester.

The aforesaid esters can be added in a quantity of up to 50 wt %, by preference in a quantity from 0.5 to 30 wt %, particularly preferably in a quantity from 1 to 20 wt %, based on the total quantity of the polymerizable adhesive composition.

Further suitable plasticizers are, for example, esters such as abietic acid esters, adipic acid esters, azelaic acid esters, benzoic acid esters, butyric acid esters, acetic acid esters, esters of higher fatty acids having approximately 8 to approximately 44 C atoms, esters of fatty acids that are epoxidized or carry OH groups, fatty acid esters and fats, glycolic acid esters, phosphoric acid esters, phthalic acid esters, linear or branched alcohols containing from 1 to 12 C atoms, propionic acid esters, sebacic acid esters, sulfonic acid esters, thiobutyric acid esters, trimellitic acid esters, citric acid esters, and mixtures of two or more thereof. Particularly suitable are the asymmetrical esters of difunctional aliphatic or aromatic dicarboxylic acids, for example the esterification product of adipic acid monooctyl ester with 2-ethylhexanol (Edenol DOA, Cognis, Düsseldorf or the esterification product of phthalic acid with butanol.

Also suitable as plasticizers are the pure or mixed ethers of monofunctional linear or branched C4-16 alcohols or mixtures of two or more different ethers of such alcohols, for example dioctyl ether (obtained as Cetiol OE, Cognis, Düsseldorf).

End-capped polyethylene glycols are additionally suitable as plasticizers, for example polyethylene or polypropylene glycol di-C1-4-alkyl ethers, in particular the dimethyl or diethyl ethers of diethylene glycol or dipropylene glycol, as well as mixtures of two or more thereof.

Particularly preferred plasticizers are tributyl citrate, triaryl phosphate, and acetyltributyl citrate.

It is moreover a preferred embodiment of the polymerizable adhesive composition according to the present invention when polymers are added, for example in order to increase the viscosity or vary the adhesion properties. These additives serve as thickeners and influence the rheology of the adhesive mixture in the desired fashion. The polymers may be used in a quantity from 1 to 60, in particular 10 to 50, by preference 10 to 30 wt %, based on the entire formulation. Especially suitable are polymers based on vinyl ethers, vinyl esters, esters of acrylic acid and methacrylic acid having 1 to 22 C atoms in the alcohol component, styrene, and co- and terpolymers derived therefrom with ethene, butadiene. Vinyl chloride/vinyl acetate copolymers having a vinyl chloride proportion from 50 to 95 wt % are preferred. The polymers can be present in liquid, resin-like, or even in solid form. It is particularly important that the polymers contain no contaminants from the polymerization process that inhibit curing of the cyanoacrylate-based adhesive composition.

If the polymers exhibit too high a water content, drying must be performed as applicable.

The molecular weight may vary over a broad range; it should be at least Mw=1.5 kg/mol but at most 1,000 kg/mol, since otherwise the final viscosity of the adhesive formulation is too high. Mixtures of the aforesaid polymers may also be used. In particular, the combination of low- and high-molecular-weight products has particular advantages in terms of the final viscosity of the adhesive formulation. Examples of suitable vinyl acetate-based polymers that may be cited are: Mowilith grades 20, 30, and 60, Vinnapas grades B1.5, B100, B17, B5, B500/20VL, B60, UW10, UW1, UW30, UW4, and UW50. Examples of suitable acrylate-based polymers that may be cited are: Acronal 4F and the Laromer grades 8912, PE55F, PO33F. Examples of suitable methacrylate-based polymers that may be cited are: Elvacite 2042, the Neocryl grades B 724, B999 731, B 735, B 811, B 813, B 817, and B722, Plexidon MW 134, Plexigum grades M 825, M 527, N 742, N 80, P 24, P 28, PQ 610. An example of suitable vinyl ether-based polymers that may be cited is: Lutonal A25. Cellulose derivatives and silica gel may also be used for thickening. The addition of polycyanoacrylates is especially to be emphasized.

The polymerizable adhesive composition according to the present invention can by preference contain one or more antimicrobial active substances in a quantity from usually 0.0001 to 3 wt %, by preference 0.0001 to 2 wt %, in particular 0.0002 to 1 wt %, particularly preferably 0.0002 to 0.2 wt %, extremely preferably 0.0003 to 0.1 wt %, based in each case on the total quantity of the polymerizable adhesive composition.

Antimicrobial active substances are differentiated, depending on the antimicrobial spectrum and mechanism of action, between bacteriostatics and bactericides, and fungistatics and fungicides. Important substances from these groups are, for example, benzalkonium chlorides, alkylarylsulfonates, halophenols, and phenol mercuric acetate. The terms “antimicrobial action” and “antimicrobial active substance” have, in the context of the teaching of the present invention, the meaning usual in the art. Suitable antimicrobial active substances are by preference selected from the groups of the alcohols, amines, aldehydes, antimicrobial acids and salts thereof, carboxylic acid esters, acid amides, phenols, phenol derivatives, diphenyls, diphenylalkanes, urea derivatives, oxygen and nitrogen acetals and formals, benzamidines, isothiazolines, phthalimide derivatives, pyridine derivatives, antimicrobial surface-active compounds, guanidines, antimicrobial amphoteric compounds, quinolines, 1,2-dibromo-2,4-dicyanobutane, iodo-2-propylbutyicarbamate, iodine, iodophores, peroxo compounds, halogen compounds, and any mixtures of the aforesaid.

The antimicrobial active substance is preferably selected from undecylenic acid, benzoic acid, salicylic acid, dihydroacetic acid, o-phenylphenol, N-methylmorpholinoacetonitrile (MMA), 2-benzyl-4-chlorophenol, 2,2′-methylenebis-(6-bromo-4-chlorophenol), 4,4′-di-chloro-2′-hydroxydiphenylether (diclosan), 2,4,4′-trichloro-2′-hydroxydiphenylether (triclosan), chlorhexidine, N-(4-chlorophenyl)-N-(3,4-dichlorophenyl)urea, N,N′-(1,10-decanediyldi-1-pyridinyl-4-ylidene)-bis-(1-octaneamine)dihydrochloride, N,N′-bis-(4-chlorophenyl)-3,12-diimino-2,4,11,13-tetraazatetradecanediimideamide, glucoprotamines, antimicrobial surface-active quaternary compounds, guanidines including the bi- and polyguanidines such as, for example, 1,6-bis-(2-ethylhexylbiguanidohexane)dihydrochloride, 1,6-di-(N1,N1′-phenyldiguanido-N5,N5′-)hexane tetrahydrochloride, 1,6-di-(N1,N1′-phenyl-N1,N1-methyldiguanido-N5,N5′-)hexane dihydrochloride, 1,6-di-(N1,N1′-o-chlorophenyldiguanido-N5,N5′-)hexane dihydrochloride, 1,6-di-(N1,N1′-2,6-dichlorophenyidiguanido-N5,N5′-)hexane dihydrochloride, 1,6-di-[N1,N1′-beta-(p-methoxyphenyl)diguanido-N5,N5′-]hexane dihydrochloride, 1,6-di-(N1,N1′-alpha-methyl-beta-phenyldiguanido-N5,N5′-)hexane dihydrochloride, 1,6-di-(N1,N1′-p-nitrophenyldiguanido-N5,N5′-)hexane dihydrochloride, omega:omega-di-(N1,N1′-phenyldiguanido-N5,N5′-)di-n-propyl ether dihydrochloride, omega:omega′-di-(N1,N1′-p-chlorophenyldiguanido-N5,N5′-)di-n-propyl ether tetrahydrochloride, 1,6-di-(N1,N1′-2,4-dichlorophenyldiguanido-N5,N5′-)hexane tetrahydrochloride, 1,6-di-(N1,N1′-p-methylphenyldiguanido-N5,N5′-)hexane dihydrochloride, 1,6-di-(N1,N1′-2,4,5-trichlorophenyldiguanido-N5,N5′-)hexane tetrahydrochloride, 1,6-di-[N1,N1′-alpha-(p-chlorophenyl)ethyldiguanido-N5,N5′-)hexane dihydrochloride, omega-omega-di-(N1,N1′-p-chlorophenyldiguanido-N5,N5′-)m-xylene dihydrochloride, 1,12-di-(N1,N1′-p-chlorophenyldiguanido-N5,N5′-)dodecane dihydrochloride, 1,10-di-(N1,N1′-phenyldiguanido-N5,N5′-)decane tetrahydrochloride, 1,12-di-(N1,N1′-phenyldiguanido-N5,N5′-)dodecane tetrahydrochloride, 1,6-di-(N1,N1′-o-chlorophenyldiguanido-N5,N5′-)hexane dihydrochloride, 1,6-di-(N1,N1′-o-chlorophenyldiguanido-N5,N5′-)hexane tetrahydrochloride, ethylenebis-(1-tolylbiguanide), ethylenebis-(p-tolylbiguanide), ethylenebis-(3,5-dimethylphenylbiguanide), ethylenebis-(p-tert-amylphenylbiguanide), ethylenebis-(nonylphenylbiguanide), ethylenebis-(phenylbiguanide), ethylenebis-(N-butylphenylbiguanide), ethylenebis-(2,5-diethoxyphenylbiguanide), ethylenebis-(2,4-dimethylphenylbiguanide), ethylenebis-(o-diphenylbiguanide), ethylenebis-(mixed amylnaphthylbiguanide), N-butylethylenebis-(phenylbiguanide), trimethylenebis(o-tolylbiguanide), N-butyltrimethylenebis-(phenylbiguanide), and the corresponding salts such as acetates, gluconates, hydrochlorides, hydrobromides, citrates, bisulfites, fluorides, polymaleates, n-cocosalkylsarcosinates, phosphites, hypophosphites, perfluoroctanoates, silicates, sorbates, salicylates, maleates, tartrates, fumarates, ethylendiamintetraacetates, iminodiacetates, cinnamates, thiocyanates, arginates, pyromellitates, tetracarboxybutyrates, benzoates, glutarates, monofluorphosphates, perfluorpropionates, and any mixtures thereof. Also suitable are halogenated xylene and cresol derivatives such as p-chlorometacresol or p-chlorometaxylene, as well as natural antimicrobial active substances of vegetable origin (e.g. from spices or herbs), or animal or microbial origin. It is preferable to use antimicrobially active surface-active quaternary compounds, a natural antimicrobial active substance of vegetable origin, and/or a natural antimicrobial active substance of animal origin, extremely preferably at least one natural antimicrobial active substance of vegetable origin from the group encompassing caffeine, theobromine, and theophylline, as well as essential oils such as eugenol, thymol, and geraniol, and/or at least one natural antimicrobial active substance of animal origin from the group encompassing enzymes such as protein from milk, lysozyme, and lactoperoxidase, and/or at least one antimicrobially acting surface-active quaternary compound having an ammonium, sulfonium, phosphonium, iodonium, or arsonium group, peroxo compounds, and chlorine compounds. Substances of microbial origin (so-called bacteriozines) may also be used. Glycine, glycine derivatives, formaldehyde, compounds that readily release formaldehyde, formic acid, and peroxides are used by preference.

Quaternary ammonium compounds (QACs) are also particularly preferred as antimicrobial active substances. The quaternary ammonium compounds (QACs) have the general formula (R1)(R2)(R3)(R4)N+X—, in which R1 to R4 represent identical or different C1-C22 alkyl radicals, C7-C28 aralkyl radicals, or heterocyclic radicals, two or (in the case of an aromatic bond such as in pyridine) even three radicals forming the heterocycle together with the nitrogen atom, for example a pyridinium or imidazolinium compound; and X— are halide ions, sulfate ions, hydroxide ions, or similar anions. For an optimum antimicrobial action, at least one of the radicals by preference has a chain length from 8 to 18, in particular 12 to 16, C atoms.

QACs can be produced by the reaction of tertiary amines with alkylating agents such as, for example, methyl chloride, benzyl chloride, dimethyl sulfate, dodecyl bromide, but also ethylene oxide. The alkylation of tertiary amines having a long alkyl radical and two methyl groups is particularly easy; in addition, the quaternization of tertiary amines having two long radicals and one methyl group can also be carried out using methyl chloride under mild conditions. Amines that possess three long alkyl radicals or hydroxy-substituted alkyl radicals are less reactive, and are preferably quaternized using dimethyl sulfate.

Suitable QACs are, for example, benzalkonium chloride (N-alkyl-N,N-dimethylbenzylammonium chloride, CAS No. 8001-54-5), benzalkon B (m,p-dichlorobenzyldimethyl-C12-alkylammonium chloride, CAS No. 58390-78-6), benzoxonium chloride(benzyldodecyl-bis-(2-hydroxyethyl)ammonium chloride), cetrimonium bromide(N-hexadecyl-N,N-trimethylammonium bromide, CAS No. 57-09-0), benzetonium chloride(N,N-dimethyl-N-[2-[2-[p-(1,1,3,3-tetramethylbutyl)phenoxy]ethoxy]ethyl]benzylammonium chloride, CAS No. 121-54-0), dialkyldimethylammonium chlorides such as di-n-decyldimethylammonium chloride (CAS No. 7173-51-5-5), didecyldimethylammonium bromide (CAS No. 2390-68-3), dioctyldimethylammonium chloride, 1-cetylpyridinium chloride (CAS No. 123-03-5), and thiazoline iodide (CAS No. 15764-48-1), as well as mixtures thereof. Particularly preferred QACs are the benzalkonium chlorides having C8-C18 alkyl radicals, in particular C12-C14 alkylbenzyldimethylammonium chloride.

Benzalkonium halides and/or substituted benzalkonium halides are obtainable commercially, for example, as Barquat® from Lonza, Marquat® from Mason, Variquat® from Witco/Sherex, and Hyamine® from Lonza, as well as Bardac® from Lonza. Further commercially obtainable antimicrobial active substances are N-(3-chlorallyl)hexaminium chloride such as Dowicide® and Dowicil® from Dow, benzethonium chloride such as Hyamine® 1622 from Rohm & Haas, methylbenzethonium chloride such as Hyamine® 10X from Rohm & Haas, and cetylpyridinium chloride such as Cepacol chloride from Merrell Labs.

Suitable thixotroping agents are known to one skilled in the art and include the following but are not limited thereto, namely silica gels, such as those that have been treated with silyl isocyanate. Examples of suitable thixotroping agents are disclosed, for example, in U.S. Pat. No. 4,720,513.

In a further preferred embodiment, the polymerizable adhesive composition according to the present invention can contain one or more skin-care active substances. Skin-care active substances may be, in particular, those agents that impart a sensory advantage to the skin, for example by delivering lipids and/or moisturizing factors to it and thus assisting healing of the affected tissue portion.

Skin-care active substances are known to one skilled in the art and can preferably be selected from the following substance groups or from mixtures of the following substance groups, although without being limited thereto:

a) Waxes such as, for example, carnauba, spermaceti, beeswax, lanolin, and/or derivatives thereof, and others.

b) Hydrophobic plant extracts.

c) Hydrocarbons such as, for example, squalenes and/or squalanes.

d) Higher fatty acids, by preference those having at least 12 carbon atoms, for example, lauric acid, stearic acid, behenic acid, myristic acid, palmitic acid, oleic acid, linoleic acid, linolenic acid, isostearic acid and/or polyunsaturated fatty acids, and others.

e) Higher fatty alcohols, by preference those having at least 12 carbon atoms, for example, lauryl alcohol, cetyl alcohol, stearyl alcohol, oleyl alcohol, behenyl alcohol, cholesterol, and/or 2-hexadecanol, and others.

f) Esters, by preference those such as cetyl octanoates, lauryl lactates, myristyl lactates, cetyl lactates, isopropyl myristates, myristyl myristates, isopropyl palmitates, isopropyl adipates, butyl stearates, decyl oleates, cholesterol isostearates, glycerol monostearates, glycerol distearates, glycerol tristearates, alkyl lactates, alkyl citrates, and/or alkyl tartrates, and others.

g) Lipids such as, for example, cholesterol, ceramides, and/or sucrose esters, and others.

h) Vitamins such as, for example, vitamins A and E, vitamin alkyl esters including vitamin C alkyl esters, and others.

i) Sun protection agents.

j) Phospholipids.

k) Derivatives of alpha-hydroxy acids.

l) Germicides for cosmetic use, both synthetic such as, for example, salicylic acid and/or others, and natural such as, for example, neem oil and/or others.

m) Silicones.

In a further preferred embodiment, the polymerizable adhesive composition may contain perfumes as a further component. Suitable perfumes are known to one skilled in the art.

In a preferred embodiment of the polymerizable adhesive composition according to the present invention, it may also contain at least one biocompatible agent, which acts to reduce the active formaldehyde concentration produced during biodegradation of the polymer in vivo (also referred to here as an “agent for reducing the active formaldehyde concentration”). The quantity will depend on the type of agent for reducing the active formaldehyde concentration and is easily determined by one skilled in the art without excessive experimentation.

A further subject of the present invention is a method for manufacturing a cyanoacrylate component, the following steps being performed in the order given:
(a) Thermal cracking of a cyanoacrylate prepolymer in the presence of at least one inorganic acid as a primary anionic polymerization inhibitor and at least one organic acid as a secondary anionic polymerization inhibitor, wherein said sulfonic acid is described by the general formula (II):

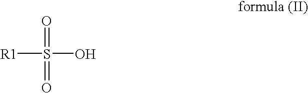

and R1 stands for an unsubstituted or mono-, di-, tri-, tetra- or penta-substituted aryl group.
(b) Separation of the resulting, preferably monomeric cyanoacrylate from the anionic polymerization inhibitor according to formula (I), by a suitable physical method, the boiling point of the resulting, preferably monomeric cyanoacrylate being below the boiling point of the at least one secondary anionic polymerization inhibitor, and the separation according to formula (I) of the resulting, preferably monomeric cyanoacrylate from the anionic polymerization inhibitor being performed by distillation at normal or reduced pressure.

The relevant boiling points in this context are preferably to be regarded as the boiling points of the individual components at normal pressure.

Through controlled coordination of the boiling points, the efficiency of the distillation process is increased, because in this way the polymerization inhibitor that is used is much more effectively separable from the respective cyanoacrylate. Overstabilization of the polymerizable adhesive composition with respect to its polymerization properties is avoided in this way, since the residual organic acid concentration in the monomer thereby obtained is much lower than is the case with conventional methods. Purification steps to be performed on the polymerizable adhesive composition immediately before use, are therefore less necessary than the addition of polymerization initiators or promoters as additives.

The term “cyanoacrylate prepolymer” is preferably understood in the sense of the present invention to refer to the product of the reaction of a cyanoacetate derivative with formaldehyde, preferably in the presence of a basic catalyst. In the course of the aforementioned reaction, cyanoacrylate prepolymers of different chain lengths and different molecular weights are formed and are accessible to thermal depolymerization.

In an especially preferred embodiment of the aforementioned process, R1 in formula (II) is described by the general formula (III), R2 being a hydrogen atom, a halogen atom, a substituted heteroatom, a substituted or unsubstituted, straight-chain, branched or cyclic alkyl chain having 1 to 10 C atoms, or including an aromatic group and/or acyl group.

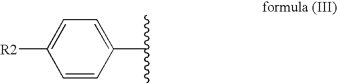

A “heteroatom” is understood to be any atom except carbon or hydrogen.

R2 preferably stands for a methyl, methoxy, ethyl, ethoxy, n-propyl, isopropyl or n-butyl group, in particular for a methyl group.

The primary anionic polymerization inhibitor can may preferably be an oxoacid, halogen acid or Lewis acid or a combination of said acids. Particularly preferred exemplary embodiments contain but are not limited to sulfur dioxide (SO₂), boron trifluoride (BF₃), nitrous oxide (N₂O), hydrogen fluoride (HF), hydrochloric acid (HCl), sulfuric acid (H₂SO₄), phosphoric acid (H₃PO₄), perchloric acid (HClO₄), or phosphorus pentoxide (P₂O₅), or combinations of said acids.

In a particularly preferred embodiment of the method according to the present invention, the at least one inorganic acid as a primary anionic polymerization inhibitor in thermal cracking of the cyanoacrylate prepolymer is present in a concentration of 800 to 35,000 ppm, particularly in a concentration of 2000 to 34,000 ppm and most preferably in a concentration of 29,000 to 33,000 ppm.

In a further preferred embodiment of the method according to the present invention, organic sulfonic acid as a secondary anionic polymerization inhibitor in thermal cracking of the cyanoacrylate prepolymer is present in a concentration of 10 to 2000 ppm, in particular in a concentration of 100 to 1000 ppm and most preferably in a concentration of 500 to 800 ppm.

In a preferred embodiment of the method according to the present invention, the residual concentration of the secondary anionic polymerization inhibitor in the resulting, preferably monomeric cyanoacrylate of the general formula (I) or in a mixture of various cyanoacrylates of the general formula (I) amounts to less than 150 ppm, preferably less than 140 ppm, 130 ppm, 120 ppm, 110 ppm, 100 ppm, particularly preferably less than 90 ppm, 80 ppm, 70 ppm, 60 ppm, 50 ppm, most especially preferably less than 40 ppm, 30 ppm, 20 ppm and most preferably 10 ppm.

Also a subject of the present patent application is a polymerizable adhesive composition according to the present invention for topical and/or internal application to mammals, in particular for medical application to their tissue, as well as the use of the polymerizable adhesive composition according to the present invention for manufacturing a pharmaceutical composition for topical and/or internal application to mammals, in particular for medical application to their tissue.

In a preferred embodiment of the present invention, the aforementioned tissue is human skin and/or the aforementioned tissue is surgically incised or traumatically lacerated tissue; the polymerizable adhesive composition according to the present invention is preferably applied to cover or close a wound.

Regardless of its inherent bacteriostatic action, the polymerizable adhesive composition according to the present invention may be sterilized directly after production and/or packaging by using a method selected from heat, ultrafiltration, and radiation, for example, or a combination of the aforementioned methods.

Another object of the present invention is a process for synthesis of a compound of the general formula (Ia):

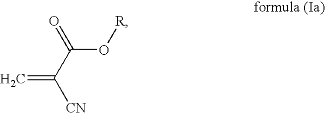

where R is a substituted or unsubstituted, straight-chain, branched or cyclic alkyl group having 5 to 18 C atoms and/or an aromatic group or acyl group, including the steps:
(a) Thermal cracking of a cyanoacrylate prepolymer in the presence of at least one inorganic acid as a primary anionic polymerization inhibitor and at least one organic acid as a secondary anionic polymerization inhibitor, said sulfonic acid being described by the general formula (II):

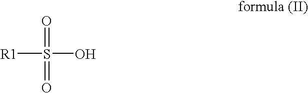

and R1 standing for an unsubstituted or mono-, di-, tri-, tetra- or pentasubstituted aryl group.
(b) Separating the resulting, preferably monomeric cyanoacrylate of the general formula (Ia) from the primary and secondary anionic polymerization inhibitors by distillation, the latter being performed at normal or reduced pressure.

The term “cyanoacrylate prepolymer” is preferably understood in the sense of the present invention to refer to the product of the reaction of a cyanoacetate derivative with formaldehyde, preferably in the presence of a basic catalyst. In the course of the aforementioned reaction, cyanoacrylate prepolymers of different chain lengths and different molecular weights are formed and are accessible to thermal depolymerization.

Preferred embodiments of general formula (Ia) include but are not limited to n-pentyl 2-cyanoacrylate, isopentyl 2-cyanoacrylate (such as 1-pentyl, 2-pentyl, and 3-pentyl), cyclopentyl 2-cyanoacrylate, n-hexyl 2-cyanoacrylate, isohexyl 2 cyanoacrylate (such as 1-hexyl, 2-hexyl, 3-hexyl, and 4-hexyl), cyclohexyl 2 cyanoacrylate, n-heptyl 2-cyanoacrylate, isoheptyl 2-cyanoacrylate (such as 1-heptyl, 2-heptyl, 3-heptyl, and 4-heptyl), cycloheptyl 2-cyanoacrylate, n-octyl 2-cyanoacrylate, 1-octyl 2-cyanoacrylate, 2-octyl 2-cyanoacrylate, 3-octyl 2 cyanoacrylate, 4-octyl 2-cyanoacrylate, decyl 2-cyanoacrylate, dodecyl 2 cyanoacrylate. Cyanoacrylates of general formula (Ia) that are preferred in particular are n-octyl 2-cyanoacrylate and 2-octyl-cyanoacrylate. Mixtures of the aforementioned cyanoacrylates are also preferred.

In a preferred embodiment of the present invention, the cyanoacrylates of general formula (I) according to the present invention may also be present in essentially monomeric form, i.e., the proportion of the corresponding polymer and/or oligomer is less than 5 wt %, preferably less than 1 wt %, and most preferably less than 0.1 wt %, each based on the total amount of inventive cyanoacrylates of general formula (Ia).

In a particularly preferred embodiment of the aforementioned method, R1 in formula (II) is described by the general formula (III), where R2 contains a hydrogen atom, a halogen atom, a substituted heteroatom, a substituted or unsubstituted, straight-chain, branched, or cyclic alkyl chain having 1 to 10 C atoms, or an aromatic group and/or acyl group.

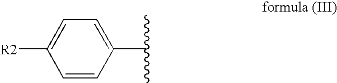

A “heteroatom” is understood to be any atom except carbon or hydrogen.

Particularly preferably R2 stands for a methyl, methoxy, ethyl, ethoxy, n-propyl, isopropyl, or n-butyl group, in particular for a methyl group.

The primary anionic polymerization inhibitor can may preferably be an oxoacid, halogen acid or Lewis acid or a combination of the aforementioned acids. Particularly preferred exemplary embodiments contain but are not limited to sulfur dioxide (SO₂), boron trifluoride (BF₃), nitrous oxide (N₂O), hydrogen fluoride (HF), hydrochloric acid (HCl), sulfuric acid (H₂SO₄), phosphoric acid (H₃PO₄), perchloric acid (HClO₄) or phosphorus pentoxide (P₂O₆), or combinations of said acids.

In a particularly preferred embodiment of the method according to the present invention, the at least one inorganic acid as a primary anionic polymerization inhibitor in thermal cracking of the cyanoacrylate prepolymer is present in a concentration of 800 to 35,000 ppm, particularly in a concentration of 2000 to 34,000 ppm and most preferably in a concentration of 29,000 to 33,000 ppm.

In a further preferred embodiment of the method according to the present invention, organic sulfonic acid as a secondary anionic polymerization inhibitor in thermal cracking of the cyanoacrylate prepolymer is present in a concentration of 10 to 2000 ppm, in particular in a concentration of 100 to 1000 ppm and most preferably in a concentration of 500 to 800 ppm.

In a preferred embodiment of the method according to the present invention, the at least one organic sulfuric acid present as the secondary anionic polymerization inhibitor in thermal cracking of the cyanoacrylate prepolymer is present in a concentration of 10 to 2000 ppm, in particular in a concentration of 100 to 1000 ppm and most preferably in a concentration of 500 to 800 ppm.

It is also preferred that in separation of the resulting, preferably monomeric compound of general formula (Ia) from the anionic polymerization inhibitor by distillation, the boiling point of the resulting, preferably monomeric compound of general formula (Ia) is below the boiling point of the secondary anionic polymerization inhibitor.

The relevant boiling points in this context are to be regarded as the boiling points at normal pressure.

Through controlled coordination of the boiling points, the efficiency of the distillation process is increased, because in this way the polymerization inhibitor that is used may be separated much more effectively from the respective, preferably monomeric compound of general formula (Ia). Any overstabilization of the preferably monomeric compound of the general formula (Ia) is prevented because the residual concentration of the secondary anionic polymerization inhibitor according to the present invention in the preferably monomeric compound of the general formula (Ia) is much lower than is the case with the traditional method.

In a preferred embodiment of the method according to the present invention, the residual concentration of the at least one organic sulfonic acid according to the present invention as a secondary anionic polymerization inhibitor in the resulting, preferably monomeric compound of the general formula (Ia) or in a mixture of various compounds of general formula (Ia) is less than 150 ppm, preferably less than 140 ppm, 130 ppm, 120 ppm, 110 ppm, 100 ppm, particularly preferably less than 90 ppm, 80 ppm, 70 ppm, 60 ppm, 50 ppm, very particularly preferably less than 40 ppm, 30 ppm, 20 ppm, and most preferably less than 10 ppm.

In a most preferred embodiment of the method according to the present invention, the curing of the resulting, preferably monomeric compound of the general formula (Ia) on an ABS surface takes place without the addition of a polymerization initiator and/or polymerization accelerator in less than 80 s, preferably in at most 50 s, most preferably in at most 25 s and most particularly preferably in at most 15 s.

The moment of curing is determined by the method described above.

The adhesive shear strength of the resulting, preferably monomeric compound of the general formula (Ia) on nylon, after curing of the aforementioned cyanoacrylate, is at least 1.6 N/mm², particularly preferably at least 1.8 N/mm² and most particularly preferably at least 2.0 N/mm².

The adhesive shear strength is determined by the method described above.

### EXEMPLIFYING EMBODIMENTS

### Example 1

2-Octyl cyanoacetate is reacted with an equimolar quantity of formaldehyde in the presence of a basic catalyst. Once the condensation reaction has ended, the solvent is removed and phosphoric acid and p-toluenesulfonic acid are added. Thermal depolymerization of the prepolymer is then accomplished, the collection vessel containing a stock solution of sulfuric acid. The monomeric crude product is additionally stabilized by adding butylhydroxyanisole (BHA) and BF₃ from a stock solution of BF₃×2H₂O, and then purified by distillation, stabilization of the monomer in the collection vessel being accomplished using a suitable quantity of SO₂ and BHA. 2-Octyl cyanoacrylate is obtained at high purity; it cures under said conditions on an ABS surface in 45 s, and its adhesive shear strength on nylon under said conditions is 2.3 N/mm².

Comparative Example 2 shows the change in adhesive properties when methanesulfonic acid is used as a comparatively volatile secondary anionic polymerization inhibitor, under otherwise identical conditions:

### Comparative Example 2

2-Octyl cyanoacetate is reacted with an equimolar quantity of formaldehyde in the presence of a basic catalyst. Once the condensation reaction has ended, the solvent is removed and phosphoric acid and methanesulfonic acid are added. Thermal depolymerization of the prepolymer is then accomplished, the collection vessel containing a stock solution of methanesulfonic acid. The monomeric crude product is additionally stabilized by adding butylhydroxyanisole (BHA) and BF₃ from a stock solution of BF₃×2H₂O, and then purified by distillation, stabilization of the monomer in the collection vessel being accomplished using a suitable quantity of SO₂ and BHA. A 2-octyl cyanoacrylate is obtained that cures under said conditions on an ABS surface in 120 s, and that has an adhesive shear strength on nylon under said conditions of 0.34 N/mm².

### Example 3

Example 3 shows the physical properties of certain cyanoacrylate components that were represented in a method analogous to Example 1.

| Weight | Adhesive shear | Adhesive shear | Curing | Curing |
|---|---|---|---|---|
| proportion^{[1]} | strength^{[3]} | strength^{[4]} | time^{[5]} | time^{[6]} |
| CA^{[2]} | on nylon | on nylon | on ABS | on ABS |
| [%] | [N/mm²] | [N/mm²] | [s] | [s] |
| 93% 2-Octyl-CA | 1.72 | 2.06 | 45 | 50 |
| 6% n-Butyl-CA | | | | |
| 98% 2-Octyl-CA | 1.93 | 1.82 | 45 | 75 |
| 99% 2-Octyl-CA | 2.57 | 2.01 | 15 | 35 |

^{[1]}Based on the total quantity of the cyanoacrylate component;
^{[2]}Cyanoacrylates (CA) according to formula (I);
^{[3]}Adhesive shear strength of a NON-STERILE cyanoacrylate component;
^{[4]}Adhesive shear strength of a STERILE cyanoacrylate component;
^{[5]}Curing time of a NON-STERILE cyanoacrylate component;
^{[6]}Curing time of a STERILE cyanoacrylate component. The determination of the adhesive shear strength and the curing time occurs under said conditions.

1. A method for manufacturing a cyanoacrylate component for utilization in adhesives, wherein the cyanoacrylate component contains a cyanoacrylate according to formula (I) or a mixture of a cyanoacrylate according to formula (I) with further cyanoacrylates according to formula (I), and curing of the sterile or non-sterile cyanoacrylate component on an ABS surface without addition of a polymerization initiator or polymerization accelerator, determined by application of a tensile force of 1 kg for at least 5 s, occurs in less than 80 s, the proportion of cyanoacrylate according to formula (I) constituting at least 90 wt % based on the total quantity of the cyanoacrylate component, and R being a substituted or unsubstituted, straight-chain, branched or cyclic alkyl group that encompasses 5 to 18 C atoms, and/or contains an aromatic group or acyl group;
the method encompassing the steps of:
(a) thermal cracking of a cyanoacrylate prepolymer in the presence of at least one inorganic acid as a primary anionic polymerization inhibitor and of at least one organic sulfonic acid as a secondary anionic polymerization inhibitor; said sulfonic acid being described by the general formula (II) 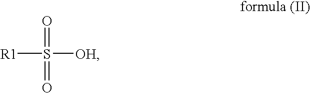
and R1 standing for an unsubstituted or a mono-, di-, tri-, tetra-, or pentasubstituted aryl group;
(b) separation of the resulting, preferably monomeric cyanoacrylate according to formula (I) from the anionic polymerization inhibitor by way of a suitable physical method, the boiling point of the resulting, preferably monomeric cyanoacrylate being below the boiling point of the secondary anionic polymerization inhibitor, and separating the resulting, preferably monomeric cyanoacrylate from the anionic polymerization inhibitor occurring by distillation at normal or reduced pressure.

2. The method of claim 1, wherein R1 is described by the general formula (III) 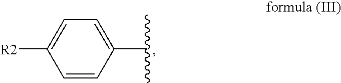
R2 containing a hydrogen atom, a substituted heteroatom, a substituted or unsubstituted, straight-chain, branched, or cyclic alkyl chain that encompasses 1 to 10 C atoms, or an aromatic group and/or acyl group.

3. The method of claim 2, wherein R2 is selected from the following groups: methyl, methoxy, ethyl, ethoxy, n-propyl, isopropyl, or n-butyl.

4. The method of claim 1, wherein the primary anionic polymerization inhibitor is an oxoacid, halogen acid, or Lewis acid, or a combination of the aforesaid acids.

5. The method of claim 4, wherein the primary anionic polymerization inhibitor is selected from sulfur dioxide, boron trifluoride, dinitrogen monoxide, hydrogen fluoride, hydrochloric acid, sulfuric acid, phosphoric acid, perchloric acid, or phosphorus pentoxide, or combinations of the aforesaid acids.

6. A method for manufacturing a compound of general formula (Ia) 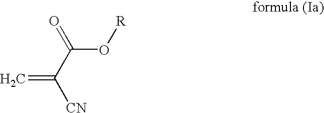
where R is a substituted or unsubstituted, straight-chain, branched or cyclic alkyl group having 5 to 18 C atoms and/or an aromatic group or acyl group, including the steps:
(a) Thermal cracking of a cyanoacrylate prepolymer in the presence of at least one inorganic acid as a primary anionic polymerization inhibitor and at least one organic sulfonic acid as a secondary anionic polymerization inhibitor; said sulfonic acid being described by the general formula (II) 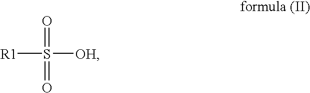
and R1 standing for an unsubstituted or a mono-, di-, tri-, tetra-, or pentasubstituted aryl group;
(b) Separating the resulting, preferably monomeric compound according to formula (Ia) from the primary and secondary anionic polymerization inhibitors by distillation, the latter being performed at normal or reduced pressure.

7. The method of claim 6, wherein the residual concentration of the at least one organic sulfonic acid as a secondary anionic polymerization inhibitor in the resulting compound of the general formula (Ia) is less than 150 ppm.

8. The method of claim 6, wherein R1 is described by the general formula (III) 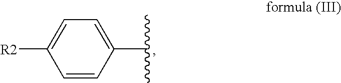
R2 containing a hydrogen atom, a substituted heteroatom, a substituted or unsubstituted, straight-chain, branched, or cyclic alkyl chain that encompasses 1 to 10 C atoms, or an aromatic group and/or acyl group.

9. The method of claim 8, wherein R2 is selected from the following groups: methyl, methoxy, ethyl, ethoxy, n-propyl, isopropyl, or n-butyl.

10. The method of claim 6, wherein the primary anionic polymerization inhibitor is an oxoacid, halogen acid, or Lewis acid, or a combination of the aforesaid acids.

11. The method of claim 10, wherein the primary anionic polymerization inhibitor is selected from sulfur dioxide (SO₂), boron trifluoride (BF₃), nitrous oxide (N₂O), hydrogen fluoride (HF), hydrochloric acid (HCl), sulfuric acid (H₂SO₄), phosphoric acid (H₃PO₄), perchloric acid (HClO₄), or phosphorus pentoxide (P₂O₅), or combinations of said acids.
